(19) United States
(12) Patent Application Publication
(10) Pub. No.: US 2025/0320540 A1
(43) Pub. Date: Oct. 16, 2025

(54) Point-of-Care Device for the Determination of Creatine Phosphokinase (CPK) in Biological Samples

(71) Applicant: In Vitro Diagnostic Solutions, Mount Laurel, NJ (US)

(72) Inventors: Robert Harper, Mount Laurel, NJ (US); George Temeng, Mount Laurel, NJ (US); Jordan T. Seville, Mount Laurel, NJ (US); Matthew DiCerbo, Mount Laurel, NJ (US)

(21) Appl. No.: 19/250,306

(22) Filed: Jun. 26, 2025

Related U.S. Application Data
(63) Continuation-in-part of application No. PCT/US2023/083965, filed on Dec. 14, 2023.
(60) Provisional application No. 63/436,213, filed on Dec. 30, 2022.

(51) Int. Cl. C12Q 1/50; B01L 3/00; G01N 21/77; G01N 21/78; G01N 21/84; G01N 33/543

(52) U.S. Cl. CPC C12Q 1/50; B01L 3/5023; B01L 3/502753; G01N 21/78; G01N 21/8483; G01N 33/54391; C12Y 207/03002; G01N 2021/775

(57) ABSTRACT

Devices, kits, and methods for testing and monitoring creatine phosphokinase (CPK) in biological samples are provided.

This patent application is a continuation-in-part of PCT/US2023/083965 filed Dec. 14, 2023, which claims the benefit of priority from U.S. Provisional Application Ser. No. 63/436,213 filed Dec. 30, 2022, teachings of which are incorporated herein by reference in their entirety.

### FIELD OF THE INVENTION

The present invention relates to methods and devices for testing and monitoring Creatine Phosphokinase (CPK) in biological samples.

### BACKGROUND OF THE INVENTION

Creatine phosphokinase (CPK), also known by the name creatine kinase (CK), is the enzyme that catalyzes the reaction of creatine and Adenosine 5′-triphosphate (ATP) to creatine phosphate (CP) and Adenosine 5′-diphosphate (ADP). The creatine phosphate created from this reaction is used to supply tissues and cells that require substantial amounts of ATP, such as the brain, skeletal muscles, and the heart, with their required ATP. CPK is a central controller of cellular energy homeostasis. Many conditions can cause derangement in CPK levels including, but not limited to, rhabdomyolysis, heart disease, kidney disease and even certain medications.

CPK is a compact enzyme of around 82 kDa that is found in both the cytosol and mitochondria of tissues where energy demands are high. In the cytosol, CPK is composed of two polypeptide subunits of around 42 kDa, and two types of subunits are found: M (muscle type) and B (brain type). These subunits allow the formation of three tissue-specific isoenzymes: CPK-MB (cardiac muscle), CPK-MM (skeletal muscle), and CPK-BB (brain). Typically, the ratio of subunits varies with muscle type: skeletal muscle: 98% CPK-MM and 2% CPK-MB and cardiac muscle: 70-80% CPK-MM and 20-30% CPK-MB, while the brain has predominantly CPK-BB. While mitochondrial creatine kinase is directly involved in the formation of creatine phosphate from mitochondrial ATP, cytosolic CPK regenerates ATP from ADP, using CP.

Normally, CPK is primarily present in heart tissue, skeletal muscles and the brain. Plasma CPK activity is significantly associated with blood pressure in the general population and is thought to contribute to hypertension by increasing vascular contractility and renal sodium retention.

Upon muscular injury, there is leakage of CPK into the bloodstream. Thus, CPK is indicative of muscular damage. CPK-MB is a more specific indicator of myocardial muscle damage, while CPK-MM is more indicative of skeletal muscle damage. The CPK activity in the serum of healthy people is due almost exclusively to CPK-MM activity (though small amounts of CPK-MB may be present) and is the result of physiological turnover of muscle tissue.

Patients with Alzheimer's disease and Pick's disease may have decreased CPK activity in the brain. CPK-BB activity primarily decreased in these patients, resulting in an overall decrease in total CPK activity.

CPK activity is one of the oldest markers of acute myocardial infarction (AMI). CPK activity begins to rise within 12 hours of AMI symptoms, peaks at 24 to 36 hours and normalizes after 48 to 72 hours. The issue with measuring CPK activity for AMI is that it is not specific to the heart. For example, CPK activity can increase in other conditions such as rhabdomyolysis, chronic muscle diseases, burns, and even after strenuous exercise.

Rhabdomyolysis may result from a crush injury, drug use, viral infections, and strenuous exercise. It typically presents with muscle pain and weakness alongside dark-colored urine. There is a breakdown of skeletal muscle, which leads to a release of CPK along with alanine aminotransferase (ALT), aspartate aminotransferase (AST), and electrolytes. The reason for the dark urine is due to myoglobinuria. A CPK level that increases to more than 1,000 IU/L is indicative of rhabdomyolysis; values over 5,000 IU/L indicate severe rhabdomyolysis. Patients with sickle cell trait who suddenly start a new strenuous exercise program such as spin class are also at an increased risk of rhabdomyolysis, with reported levels of CPK higher than 70,000 IU/L in some cases. The most common complication resulting from rhabdomyolysis is acute kidney injury. Therefore, any patient with suspected rhabdomyolysis should receive prompt treatment with intravenous fluids to preserve kidney function.

Serum CPK activity is also greatly elevated in all types of muscular dystrophy. In progressive muscular dystrophy (particularly Duchenne sex-linked muscular dystrophy), enzyme activity in serum is highest in infancy and childhood (7 to 10 years of age) and may be increased long before the disease is clinically apparent. Serum CPK activity characteristically falls as patients get older and as the mass of functioning muscle diminishes with the progression of the disease. About 50% to 80% of the asymptomatic female carriers of Duchenne dystrophy show three-fold to six-fold increases in CPK activity. Quite high values of CPK are noted in viral myositis, polymyositis, and similar muscle diseases. However, in neurogenic muscle diseases, such as myasthenia gravis, multiple sclerosis, poliomyelitis, and Parkinsonism, serum enzyme activity is not increased.

Serum CPK activity also demonstrates an inverse relationship with thyroid activity. About 60% of hypothyroid subjects show an average elevation of CPK activity five-fold more than the upper reference limit. The major isoenzyme present is CPK-MM, suggesting muscular involvement. Even in subclinical hypothyroidism, there is some degree of dysfunction in skeletal muscle metabolism. Strenuous, prolonged exercise will result in large increases in serum CPK activities. In untrained persons, serum CPK appears to increase proportionately to the duration and intensity of the exercise; however, conditioned persons often show smaller changes in serum CPK activity. Sustained exercise, such as in well-trained, long-distance runners, increases the CPK-MB content of skeletal muscle, owing to the phenomenon of “fetal reversion,” in which fetal patterns of protein synthesis reappear. Thus serum CK-MB isoenzyme may increase in such circumstances. See Islam et al. Int J Med Res Prof 2020 6 (3), 39-43.

Thus, the CPK-MB isoenzyme started being used in tandem mass spectrometry assays to aid in the diagnosis of AMI. Although CPK-MB is a more specific marker for heart muscle damage compared to total CPK, CPK-MB can still be elevated in conditions like severe muscle injury, congestive heart failure, and certain arrhythmias, which means interpreting a high CPK-MB level alone might not always definitively indicate a heart attack. Further, these assays are timely, expensive and require a skilled technician for performance in a clinical setting.

There is a need for simpler, faster, and more cost-effective point-of-care assays for measuring CPK enzyme activity.

### SUMMARY OF INVENTION

This disclosure relates to an electrochemical or colorimetric device, referred to herein as “CPK Now”, and methods and kits for use of this device for the quantitative determination of Creatinine Phosphokinase (CPK) in biological samples. The device comprises a combination of components designed to elicit a measurable electrochemical or colored end-product from the application of a biological sample containing CPK. The device and method requires less than 25 μL of a biological sample such as blood, saliva, or urine for CPK quantitation. The end-electrical response or color of the reagent layer is proportional to the concentration of CPK in the biological sample. The device, kits, and methods thereof provide quantitative results, and are faster, more rugged, and easier to perform than analogous wet chemistry assays, lateral flow assays, or dedicated laboratory assays. The device, kits, and methods of this disclosure can be used at the point-of-care, at home, in the hospital, or at a clinician's office to measure CPK.

### BRIEF DESCRIPTION OF THE FIGURES

FIG. 1 is a graph showing the linear regression of calculated CPK spiked values versus gravimetric spiked whole blood values using the device of this disclosure.

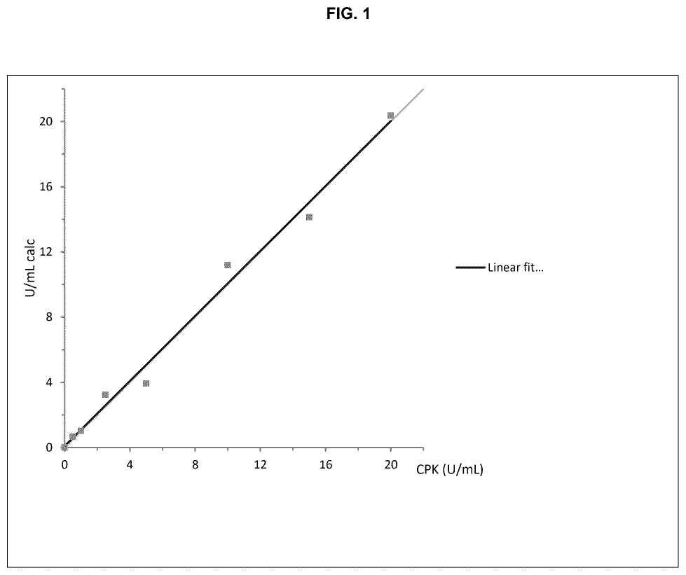

FIG. 2 is a diagram of a non-limiting embodiment of a test strip of the present invention within the top and bottom of a cassette.

FIG. 3 is a photograph showing a non-limiting embodiment of a kit inclusive of the test strips of this disclosure, means for obtaining a blood sample for testing, a portable hand-held meter for the test strips, and a cell phone application for quantitative analysis and/or transmitting data from the test strip to a health care provider.

### DETAILED DESCRIPTION OF THE INVENTION

The present invention relates to a device comprising a unique test strip for the determination of CPK in biological samples, as well as kits and methods for use of the device in measuring CPK levels in biological samples.

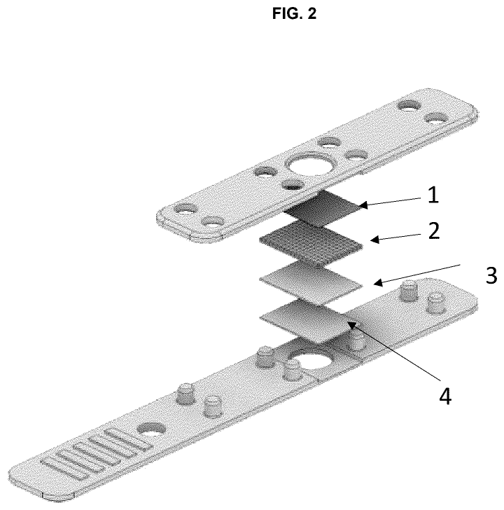

In simplest form, the test strip is comprised of a means for separating blood and a means for measuring CPK. In one non-limiting embodiment, the test strip of the present invention is comprised of four superimposed layers. The layers can be adhered to a base material through lamination with adhesives or through compression in a cassette with a top and bottom as depicted in FIG. 2, without the requirements of lamination. The test strip of the present invention is useful in quantifying CPK in a biological sample of a subject and for detecting changes in CPK levels over time in biological samples of a subject. Typically, the biological sample is whole blood taken from a subject's finger or heel-stick. In one non-limiting embodiment, the combination of layers in the test strip allows for zero percent bias in the range of 32 to 52% hematocrit and in the analytical range of 0 to 60,000 U/L CPK. Accordingly, this invention can be used for both the initial diagnosis of elevated levels of CPK associated with conditions such as, but not limited to, rhabdomyolysis and AMIs and for monitoring CPK levels in individuals in need thereof. In one non-limiting embodiment, the device is used to identify subjects with elevated CPK levels in need of prompt treatment with intravenous fluids to preserve kidney function. Examples of such subjects include, but are not limited to those on restricted diets, those suffering from damaged muscle tissue, those suffering from damage to their heart and/or kidneys and/or those on medications associated with elevated CPK levels.

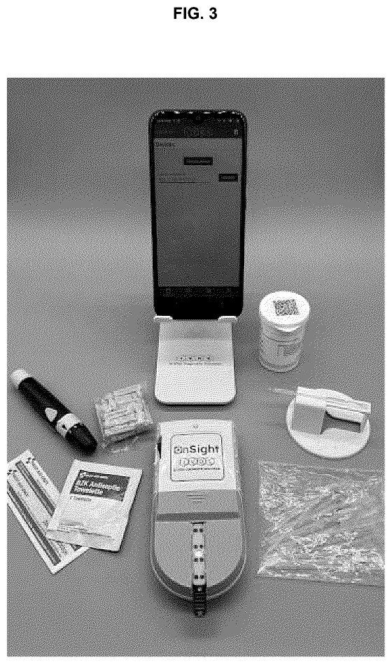

When coupled with an analyzer such as the hand-held meter and/or a computer-implemented method for capturing and using quantitative and qualitative results from point-of-collection devices for different diagnostic assays through a mobile device as depicted in FIG. 3, the device of this invention provides a point of care test (POCT) for CPK which is much faster and less expensive as compared to mass spectrometry analysis and can be used in home monitoring without any requirements for a skilled technician. Further, via remote dissemination of data to a care provider, a subject may be able to avoid a hospital visit and/or IV treatment for 3 days.

As shown in FIG. 2, in one non-limiting embodiment, the test strip comprises four layers, 3 of which are membranes. The first layer is referred to as the sample spreading layer and is labeled as 1 in FIG. 2. The sample spreading layer is capable of distributing or metering the cells in the biological sample evenly across the surface of the primary membrane. The sample spreading layer provides a uniform concentration of cells between the interface of the spreading layer and the underlying primary membrane. The spreading layer 1 can be a mesh material, an isotropically porous membrane (same porosity throughout), or an anisotropic membrane (a gradient in porosity). The spreading layer 1 can be composed of nylon or polyester with an average pore size in the range of 10-200 μm. Precise permeability of the spreading layer is critical, as it determines whether or not a homogeneous biological sample will be uniformly distributed across the surface of the underlying primary membrane layer. The surface of the spreading layer is in direct contact with the primary membrane for uniform transfer of the biological material through a lateral and vertical migration of the biological fluid.

The test strip further comprises a primary membrane layer labeled 2 in FIG. 2. Fluid of the biological sample flows transverse across the spreading layer 1 before migrating vertically into the primary membrane layer 2. The primary membrane layer is a blood separation membrane. This primary whole blood separation membrane is also referred to herein as Membrane-1. Membrane-1 contains a non-hemolytic surfactant, a hemagglutinating agent, an oxidizing agent that oxidizes hemoglobin and L-ascorbic acid to non-interfering forms, a polymer, salts, bulking agent, and a buffer buffered in the range of pH 5.0 and pH 9.0. Membrane-1 can be composed of one, or a combination of several, material(s) including, but not limited to, bound or unbound borosilicate glass microfiber, nylon, polyester, cellulose, cellulose acetate, nitrocellulose, polycarbonate, polyvinylidene difluoride, polyether sulfone, or polysulfone with an average pore size in the range of 3.0-15.0 μm. Membrane-1 is comprised of hemagglutinating agents, including but not limited to, anti-red blood cell antibodies, chitosan, poly(diallyl dimethylammonium chloride), poly(allylamine), poly(allylamine hydrochloride), poly(4-vinylpyridine), poly(2-vinylpyridine), poly(2-vinyl-1-methylpyridinium bromide), poly[bis(2-chloroethyl) ether-alt-1,3-bis[3-(dimethyl amino) propyl]urea] quaternized, hexadimethrine bromide, poly-L-lysine, poly-L-lysine hydrobromide, poly-D-lysine, poly-D-lysine hydrobromide, poly-DL-lysine hydrobromide, poly-L-arginine hydrochloride, poly(ethylenimine hydrochloride), diethylaminoethyl dextran (DEAE-dextran), diethylaminoethyl dextran chloride, poly(n,n-dimethyl-3,5-dimethylene piperidinium chloride), or unconjugated crude or purified lectins which preferably agglutinate human type O erythrocytes, or most preferably non-specifically, to an efficient degree such as those from Maclura pomifera (<5 μg/mL MPA for type O), Phaseolus vulgaris (<5 μg/ml PHA-E for type O), Ulex europaeus (<4 μg/ml UEA-I for type O), and Solanum tuberosum (<15 μg/ml STA for type O). Additionally, the lectins can also be combined with a Neuraminidase, such as those from Clostridium perfringens (C. welchii), Vibrio cholerae, Arthrobacter ureafaciens, Streptococcus pneumoniae, or recombinant derivatives thereof expressed in Escherichia coli, to increase the hemagglutination efficiency of the lectin by 10-60× in the primary blood separation membrane. The hemagglutinating agents can be immobilized together with a polymer, including but not limited to, hydroxypropyl cellulose, hydroxyethyl cellulose, poly(vinyl alcohol), dextran, diethylaminoethyl dextran (DEAE-dextran), diethylaminoethyl dextran chloride, dextran sulfate sodium salt, poly(acrylic acid), poly(sodium 4-styrenesulfonate), chitosan, λ-carrageenan, gelatin, sodium carboxymethyl cellulose, xanthan gum, polyvinyl pyrrolidone, poly(1-vinylpyrrolidone-co-vinyl acetate), poly(vinyl acetate) or poly(methyl vinyl ether-alt-maleic anhydride). Non-limiting examples of oxidizing agents that oxidize hemoglobin and L-ascorbic acid into non-interfering forms include potassium nitrite (KNO2), sodium nitrite (NaNO2), 4-hydroxy-2,2,6,6-tetramethylpiperidin-1-oxyl (4-hydroxy-TEMPO), potassium iodate (KIO3) and sodium iodate (NaIO3).

The test strip further comprises Membrane-2 labeled as layer 3 in FIG. 2 underlying Membrane-1, layer 2. The plasma and remaining cells from the primary membrane continue migrating vertically downward into the secondary membrane referred to herein as Membrane-2. T Membrane-2 is in direct contact with Membane-1. Membrane-2 is composed of one, or a combination of several, material(s) including, but not limited to, bound or unbound borosilicate glass microfiber, nylon, polyester, cellulose, cellulose acetate, nitrocellulose, polycarbonate, polyvinylidene difluoride, polyether sulfone or polysulfone with an average pore size in the range of 0.8-5.0 μm. Membrane-2 contains a non-hemolytic surfactant, polymer, bulking agents, and a buffer. The optimal pH of human CPK can be either 6.5 or 9.0 depending on the forward or reverse reaction being desirable. In one non-limiting embodiment, Membrane-2 contains an immobilized preconditioning buffer in the pH range of 5.0 to 9.0. Without being bound to any particular theory, it is believed that preconditioning of the biological fluid allows time for the homogenous mixing of the excipients while also buffering the biological fluid to a suitable pH for the enzymatic determination of CPK. In one non-limiting embodiment, Membrane-2 can also contain a native or modified L-ascorbate oxidase from either Cucurbita pepo, Cucurbita pepo var. medullosa, Cucumis sp., Acremonium sp., Trichoderma lignorum APC-9314 (FERM-P-13972), Eupenicillium brefeldianum APC-9315 (FERM P-BP-5053), Penicillium canescens IFO7955, or recombinant derivatives thereof expressed in Escherichia coli, to convert endogenous L-ascorbate into dehydroascorbate to prevent any interference in the redox detection mechanism. Additionally, in one non-limiting embodiment, Membrane-2 can also contain an oxamate salt to prevent endogenous L-lactate dehydrogenase and L-lactate from interfering in the measured redox reaction. In one non-limiting embodiment, the oxamate salt is sodium oxamate. In one non-limiting embodiment, Membrane-2 can also contain ethylene glycol-bis(β-aminoethyl ether)-N,N,N′,N′-tetraacetic acid (EGTA) to preferentially chelate endogenous calcium ions that would otherwise inhibit CPK activity via competition with magnesium ions at the active site of the enzyme. In one non-limiting embodiment, Membrane-2 contains a salt of adenosine-5′-monophosphate (AMP), a fluoride salt such as sodium fluoride or potassium fluoride, P1,P5-di(adenosine-5′) pentaphosphate, pentasodium salt (DPP), or any synergistic combination thereof to inhibit any myokinase (adenylate kinase) interference. Membrane-2 also contains the substrate Adenosine 5′-diphosphate (ADP). Other potential interferents which this assay may be still susceptible to include amikacin, amoxicillin, cephalothin, calcium dobesilate, dopamine, L-DOPA, methotrexate, nitrofurantoin, and sulfamethoxazole. In one non-limiting embodiment, the components on Membrane-2 are immobilized with a polymer. Examples of polymers include, but are not limited to, hydroxypropyl cellulose, hydroxyethyl cellulose, poly(vinyl alcohol), dextran, diethylaminoethyl dextran (DEAE-dextran), diethylaminoethyl dextran chloride, dextran sulfate sodium salt, poly(acrylic acid), poly(sodium 4-styrenesulfonate), chitosan, A-carrageenan, gelatin, sodium carboxymethyl cellulose, xanthan gum, polyvinyl pyrrolidone, poly(1-vinylpyrrolidone-co-vinyl acetate), poly(vinyl acetate) or poly(methyl vinyl ether-alt-maleic anhydride).

The test strip further comprises Membrane-3 labeled as layer 4 in FIG. 2. The buffered fluid containing CPK travels from Membrane-2 to the underlying tertiary membrane, Membrane-3. The tertiary membrane is referred to herein as “the reagent membrane” or “Membrane-3”. The reagent membrane is visually clean and smooth with submicron-sized pores, thus providing excellent optical and reflective properties. Membrane-3 is composed of one, or a combination of several, material(s) including, but not limited to, nylon, cellulose, cellulose acetate, nitrocellulose, polycarbonate, polyether sulfone or polysulfone with an average pore size in the range of 0.03-1.2 μm. This reagent membrane provides a uniform end-color in the read-zone for precise detection. In one non-limiting embodiment, the reagent membrane is treated with a buffer, surfactant, stabilizers, colorimetric redox indicator(s), cofactors, substrates, enzymes, and an electron mediator, all of which are immobilized onto the reagent membrane using a polymer. In one non-limiting embodiment, the reagent membrane for CPK detection comprises a creatine phosphate salt, glycerol, 4-aminoantipyrine, a Trinder reagent, peroxidase as an electron mediator, L-α-glycerophosphate oxidase, and glycerol kinase. In one non-limiting embodiment, the creatine phosphate salt is either a sodium or potassium salt of creatine phosphate. In one non-limiting embodiment, the peroxidase is from Horseradish (Armoracia rusticana) or recombinant derivatives thereof expressed in Escherichia coli. In one non-limiting embodiment, the glycerol kinase is from Cellulomonas sp. JCM2471, Flavobacterium meningosepticum, or recombinant derivatives thereof expressed in Escherichia coli. In one non-limiting embodiment, the L-α-glycerophosphate oxidase is from Streptococcus sp. GPOS-53, Pediococcus homari IFO 12217, or recombinant derivatives thereof expressed in Escherichia coli. In one non-limiting embodiment, the Trinder reagent is N-Ethyl-N-(2-hydroxy-3-sulfopropyl)-3-methylaniline, sodium salt, dihydrate (TOOS), N,N-Bis(4-sulfobutyl)-3-methylaniline, disodium salt (TODB), N-Ethyl-N-(3-sulfopropyl)-3-methylaniline, sodium salt, monohydrate (TOPS), or N,N-Bis(4-sulfobutyl)-3,5-dimethylaniline, disodium salt (MADB). In one non-limiting embodiment, the reagent membrane is buffered to a pH in the range of 5.0-9.0. the components on Membrane-3 are immobilized with a polymer including, but not limited to, hydroxypropyl cellulose, hydroxyethyl cellulose, poly(vinyl alcohol), dextran, diethylaminoethyl dextran (DEAE-dextran), diethylaminoethyl dextran chloride, dextran sulfate sodium salt, poly(acrylic acid), poly(sodium 4-styrenesulfonate), chitosan, A-carrageenan, gelatin, sodium carboxymethyl cellulose, xanthan gum, polyvinyl pyrrolidone, poly(1-vinylpyrrolidone-co-vinyl acetate), poly(vinyl acetate) or poly(methyl vinyl ether-alt-maleic anhydride). The biological fluid slowly migrates vertically downward onto the reagent membrane. The end-color intensity of the reagent membrane can be measured in percent reflectance units on a handheld meter and converted to U/L through a preprogrammed curve set, calibrated against a laboratory reference instrument, or as an optical image measuring RGB values calibrated against a laboratory reference instrument, or electrochemical detection. The concentration of U/L CPK can be determined by the end-color intensity at a given time or by kinetic rate determination. In one non-limiting embodiment, the reagent membrane is positioned facing a light emitting diode (LED) and photodiode to measure the end-color intensity of the reagent membrane, or positioned facing a camera to image the end-color using Red/Green/Blue (RGB) values. In one non-limiting embodiment, the LED and photodiode can detect the end-color of the generated diimine dye from the coupled Trinder reagent reaction that has a lambda max wavelength in the range of 500 nm and 700 nm for reflectance determination. Quantification of the analyte of interest can be achieved via percent reflectance versus a gold-standard reference instrument. The end-color can also be quantified using a camera to image the end-color intensity of the generated diimine dye. Quantification by image analysis can be calculated from RGB values. The concentration of CPK can also be determined electrochemically.

The CPK catalyzes the dephosphorylation of creatine phosphate and the series of sequential enzymatic steps to produce hydrogen peroxide (H2O2), which is then reduced to two water molecules (2*H2O) by peroxidase. The peroxidase utilizes the Trinder reagent and 4-Aminoantipyrine (4-AAP) as substrates and couples them together to create an oxidized diimine dye shown in thee reaction mechanism below:

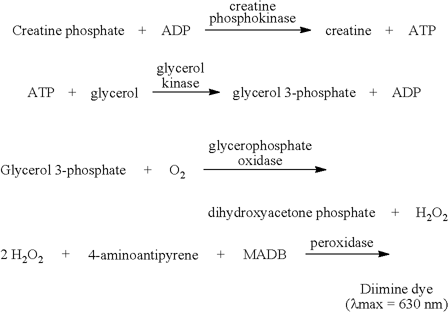

The “normal” range for CPK in the blood is 50-170 U/L. This invention demonstrates exceptional performance over the analytical range of 0 to 30,000 U/L CPK. Above 20,000 to 25,000, a subject is typically admitted to the hospital.

In practice, the test strip of the present invention determines CPK levels as a point-of-care test.

The volume of blood used in the device, using a fingerstick whole blood sample, is less than 25 μL. This will allow for ease-of-use for the patient.

Two significant contributions of this invention are the ability to detect low concentration levels of CPK with a high degree of reliability (sensitivity), and the ability to discriminate between various concentrations of CPK over the clinically significant range. This is achieved by an enzyme-coupled, colorimetric redox mechanism, where the end color (reflectance) is converted to concentration of CPK in biological solutions.

What is claimed is:

1. A device for quantitatively measuring creatinine phosphokinase (CPK) in a biological sample, said device including a test strip comprising:
a sample spreading layer;
a primary whole blood separation membrane;
a secondary whole blood separation membrane; and
a reagent membrane for CPK detection comprising Adenosine 5′-diphosphate (ADP), a creatine phosphate salt, glycerol, 4-aminoantipyrine, a colorimetric redox indicator, an electron mediator, L-α-glycerophosphate oxidase and glycerol kinase.

2. The device of claim 1, further comprising an electrochemical sensor, light emitting diode (LED) and photodiode, or camera positioned adjacent to the test strip to detect the end-color intensity of the reagent membrane indicative of the CPK concentration in the biological sample.

3. The device of claim 1, wherein the sample spreading layer comprises a nylon or polyester mesh with a pore size in the range of 10-200 μm that distributes the biological sample laterally and evenly across a surface of the primary whole blood separation membrane.

4. The device of claim 1, wherein the primary whole blood separation membrane is composed of bound or unbound borosilicate glass microfiber, nylon, polyester, cellulose, cellulose acetate, nitrocellulose, polycarbonate, polyvinylidene difluoride, polyethersulfone or polysulfone or combinations thereof, with an average particle retention size specifically in the range of 3-15 μm.

5. The device of claim 4, wherein the primary whole blood separation membrane comprises:
a buffer, a non-hemolytic surfactant, a polymer, and hemagglutinating agents to prevent the passage of red blood cells into the reagent membrane; and
an agent that both oxidizes hemoglobin to methemoglobin and oxidizes ascorbate to dehydroascorbate.

6. The device of claim 1, wherein the secondary whole blood separation membrane is composed of bound or unbound borosilicate glass microfiber, nylon, polyester, cellulose, cellulose acetate, nitrocellulose, polycarbonate, polyvinylidene difluoride, polyethersulfone or polysulfone or combinations thereof, with an average pore size in the range of 0.8-5.0 μm.

7. The device of claim 6, wherein the secondary whole blood separation membrane is treated with a preconditioning buffer at a concentration of 10-150 mM adjusted to pH 5.0-9.0 and a non-hemolytic surfactant which are both immobilized onto the membrane using a polymer.

8. The device of claim 1, wherein the reagent membrane is optically smooth and comprised of nylon, cellulose, cellulose acetate, nitrocellulose, polycarbonate, polyethersulfone, polysulfone or combinations thereof, with an average pore size in the range of 0.03-1.2 μm.

9. The device of claim 8, wherein the reagent membrane is treated with a buffer, surfactant, stabilizers, a colorimetric redox indicator, a cofactor, substrates, enzymes and an electron mediator, all of which are immobilized on the reagent membrane using a polymer.

10. The device of claim 9, wherein the reagent membrane is buffered to a pH in the range of 5.0-9.0.

11. The device of claim 9, wherein the electron mediator is peroxidase with an optimum pH activity in the range of 6.0 and 7.0.

12. The device of claim 11, wherein the peroxidase is derived from Horseradish (Armoracia rusticana) or recombinant derivatives thereof expressed in Escherichia coli.

13. The device of claim 9, wherein the colorimetric redox indicator is a Trinder reagent coupling with 4-aminoantipyrene (4-AAP) that produces a colored diimine dye upon oxidation, with the diimine dye having a lambda max wavelength between 500 nm and 700 nm.

14. The device of claim 1, wherein the layer and membranes of the test strip are adhered to a base material through lamination with adhesives or through compression in a cassette.

15. The device of claim 1, wherein the test strip allows for zero to six percent bias in the hematocrit range of 32-52% through the analytical range of 0-30,000 U/L CPK.

16. A method for quantitatively measuring creatine phosphokinase (CPK) in a biological sample, said method comprising applying the biological sample to the device of claim 1 and measuring CPK in the biological sample.

17. The method of claim 16, wherein biological sample applied is less than 25 μL.

18. The method of claim 16, wherein the reagent membrane of the test strip is positioned facing a light emitting diode (LED) and photodiode to measure the end-color intensity of the reagent membrane indicative of CPK activity in the biological sample.

19. The method of claim 16, wherein the reagent membrane of the test strip is positioned facing a camera to image the end-color and quantified using Red/Green/Blue (RGB) values.

20. A kit for detecting creatine phosphokinase (CPK) in a biological sample, said kit comprising a device of claim 1, a means for obtaining a blood sample for testing, a portable hand-held meter for the test strips, and a cell phone application for the quantitative analysis and/or transmitting of data from the test strip to a health care provider.
